(19) United States
(12) Patent Application Publication
(10) Pub. No.: US 2007/0255119 A1
(43) Pub. Date: Nov. 1, 2007

(54) Separate computing device for medical device with computing capabilities

(76) Inventors: David Mordaunt, Los Gatos, CA (US); Katrina Bell, Palo Alto, CA (US); Michael Simoneau, Morgan Hill, CA (US)

Correspondence Address: DLA PIPER US LLP, 2000 UNIVERSITY AVENUE, E. PALO ALTO, CA, 94303-2248, US

(21) Appl. No.: 11/717,505

(22) Filed: Mar. 12, 2007

Related U.S. Application Data
(60) Provisional application No. 60/782,201, filed on Mar. 13, 2006.
(60) Provisional application No. 60/857,939, filed on Nov. 8, 2006.

(51) Int. Cl. A61B 5/00

(52) U.S. Cl. 600/300

(57) ABSTRACT

A medical system that includes a medical device having a computing unit for controlling the medical device to perform a clinical procedure, a secondary computing device linked to the medical device via a first communications link, and an external resource or network linked to the secondary computing device via a second communications link. The secondary computing device is configured to provide a communications interface between the medical device and the external resource or network. The secondary computing device can include an input/output unit to access data stored on the medical device and/or the secondary computing device, and to input data relating to the clinical procedure performed by the medical device.

This application claims the benefit of U.S. Provisional Application Nos. 60/782,201, filed Mar. 13, 2006, and 60/857,939, filed Nov. 8, 2006, both of which are incorporated herein in their entirety by reference.

### FIELD OF THE INVENTION

The present invention relates to medical devices, and in particular to the control of the medical devices that have computing capabilities.

### BACKGROUND OF THE INVENTION

There are many types of therapeutic medical devices that include a computing capability using an internal/dedicated computing unit. A computing unit is a dedicated computer or a dedicated computing device with processing power and/or storage memory (e.g. CPU, microprocessor, etc.), which typically runs proprietary operating software, for operating the medical device to perform clinical (i.e. therapeutic, treatment, and/or diagnostic) procedures. The computing unit is either integral to the medical device, or is connected to the medical device in a dedicated manner to operate the medical device. Examples of such medical devices include ophthalmic diagnostic and treatment devices (e.g. photocoagulators, imaging systems, optical coherence tomography (OCT) systems, fundus cameras, etc.).

Often times, it is desirable to have the medical device communicate to external resources and networks for electronic record keeping, database access and storage, remote servicing, printing, data acquisition, etc., which increases the efficiency and functionality of the medical device. However, there are drawbacks to connecting the medical device to external resources and networks. First, having the computing unit monitor and access external resources and networks can result in the computing unit expending unacceptable processing power/bandwidth on such monitoring and access, preventing it from expending the necessary processing power/bandwidth on controlling and operating the medical device. Second, providing the computing unit with access to resources and networks and/or providing resources/networks access to the medical device, opens the medical device up to the outside world, making it more difficult to protect the device and/or its operating software from unauthorized access, exploitation and damage (e.g. computer virus).

There is a need for a medical device that can communicate with external resources and networks without compromising the performance and safety of the medical device.

### SUMMARY OF THE INVENTION

The present invention solves the aforementioned problems by providing a medical system that includes a communications interface that is separate from the computing unit of the medical devices. Specifically, the medical system includes a medical device having a computing unit for controlling the medical device to perform a clinical procedure, a secondary computing device linked to the medical device via a first communications link, and an external resource or network linked to the secondary computing device via a second communications link, wherein the secondary computing device is configured to provide a communications interface between the medical device and the external resource or network.

Other objects and features of the present invention will become apparent by a review of the specification, claims and appended figures.

### BRIEF DESCRIPTION OF THE DRAWINGS

FIG. 1 is a diagram showing the interconnections between a medical device having a computing unit, a secondary computing device, and an external resource/network.

FIG. 2 is a system diagram showing the components of an ophthalmic photocoagulator.

FIG. 3 is a diagram of a pattern P of a single spot.

FIGS. 4A-4G are diagrams of symmetrical patterns P of spots.

FIGS. 5A-5F are diagrams of non-symmetrical patterns P of spots.

FIGS. 6A-6B are diagrams of patterns P of spots with fully enclosed exclusion zones.

FIGS. 7A-7B are diagrams of patterns P of spots with partially open exclusion zones.

FIG. 8 is a front view of a graphic user interface screen for operating the ophthalmic photocoagulator.

FIG. 9 is a front view of the graphic user interface screen displaying multiple possible pattern configurations from which to choose from.

FIGS. 10-14 are front views of data screens displayed by a display of the input/output device for retrieving and viewing data for the ophthalmic photocoagulator.

FIG. 15 illustrates the various the quadrants on the eye in which patterns of spots can be used to treat the eye.

FIG. 16 is a front view of a procedure summary screen for which the physician can annotate and print to summarize a recent procedure conducted using the medical device.

### DETAILED DESCRIPTION OF THE PREFERRED EMBODIMENTS

The present invention is system architecture and method for allowing a medical device to access external resources and networks without compromising the performance and safety of the medical device operation.

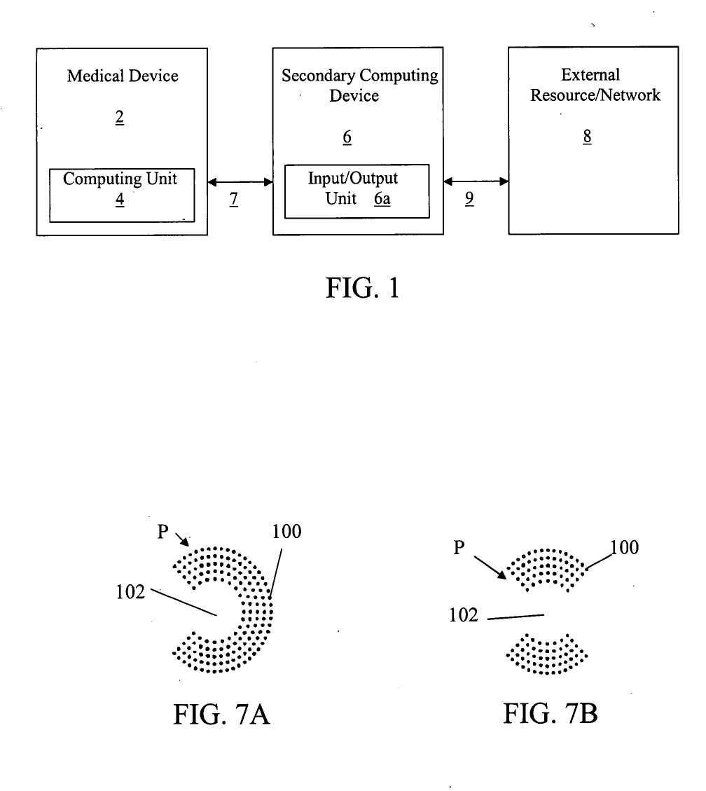

FIG. 1 illustrates the basic system architecture, where a medical device 2 (having a computing unit 4 for operating the medical device to perform a clinical procedure) is connected to a secondary computing device 6 via a two-way communications link 7. The clinical procedure can include a therapeutic, a treatment, and/or a diagnostic procedure. The secondary computing device 6 is connected to an external resource or network 8 via a two-way communications link 9, and preferably includes an optional input/output device 6a (e.g. keyboard, mouse, touch-screen display and/or visual display, etc.).

The secondary computing device 6 acts as the main communication interface between the medical device 2 and the external resource/network 8. This configuration protects the medical device hardware and software, and enables simplified communication with the medical device 2 without unnecessarily tying up the processing power of the computing unit 4. The secondary computing device 6 can be a stand alone, standardized computer (operating specialized or standardized software) which is more easily serviced and configured than the more specialized medical device computing unit 4. Medical devices usually require specialized installation by personnel specifically trained and authorized to work with medical devices. Thus, by using standardized equipment for the secondary computing device 6, it can be installed and serviced by non-medical device trained personnel, without the many precautions often necessary when working with the medical device 2 itself. An exemplary, non-limiting, simplified implementation would entail using a common stand-alone personal computer (e.g. desktop or notebook), running standard software (e.g. Microsoft Windows operating system) as the secondary computing device 6. The secondary computing device 6 can be easily upgraded as needed, again without having to go through the more rigorous exercise of upgrading a medical device including all the extra precautions associated with medical device upgrades.

The external resource or network 8 can include a closed data network such as a data network for a particular medical group, facility, or medical device manufacturer, a more open data network such as the Internet, as well as servers, printers, fax machines, visual displays, etc. Remote monitoring of the medical device 2 is now possible where the secondary computing device either probes stored data on the medical device 2, or includes information directly uploaded from the medical device 2 onto the secondary computing device 6. Medical device information can then be communicated via the external resource/network 8 (e.g. medical group network, the Internet, etc.) using any known protocol (direct data transfer, e-mail, facsimile, etc.) to provide system status and monitoring without unnecessarily tying up the processing capabilities of the medical device 2.

Additionally, for medical devices that do not store data locally, or do not allow hard drive reading, the medical device 2 can be monitored real-time by the secondary computing device 6 for activities and parameters that are accessible via an external port, where such data can be recorded by the secondary computing device and/or transmitted to the external resource/network 8 real-time or in a delayed manner.

Trouble-shooting of printers, cameras, accessories, internet-connectivity, etc. on the secondary computing device 6 is simplified because standardized operating system utilities with full access functionality can be employed. In contrast, accessories and connections on the medical device 2 can be problematic because access to the medical device operating system and functionality is often restricted to protect the integrity of the medical device software/hardware.

The addition of the secondary computing device 6 can also enhance the use of patient and treatment specific information, which can now be conveniently added to medical device treatment summaries and electronic medical records using the secondary computing device 6 and its optional input/output device 6a, to provide a complete report, without the need to add keyboards and extra software to the medical device 2.

Communications link 7 between the medical device 2 and the secondary computing device 6 can be any well known two-way link used with computing devices, such as, but not limited to, a serial connection, a wired or wireless USB connection, a phone line connection, an Ethernet connection, a wireless communications connection, etc. (or any combination of the above connections). Due to the security risks of a direct connection between the medical device and other devices, a unique customized connector could be used for link 7, such as a non-standard plug and wire, so that standard plugs or other interfaces could not be inserted to complete communications link 7. This would ensure that only certain trusted secondary computing devices 6 can be connected to the medical device 2. In addition, hardware or software firewalls could be installed in the medical device 2 or the secondary computing device 6 for added protection. Communications link 9 between the secondary computing device 6 and the external resource/network can also be any well known two-way link used with computing devices and networks, such as, but not limited to, a serial connection, a wired or wireless USB connection, a phone line connection, an Ethernet connection, a DSL or cable modem connection, a wireless communications connection, etc.

Photocoagulator Exemplary Embodiment

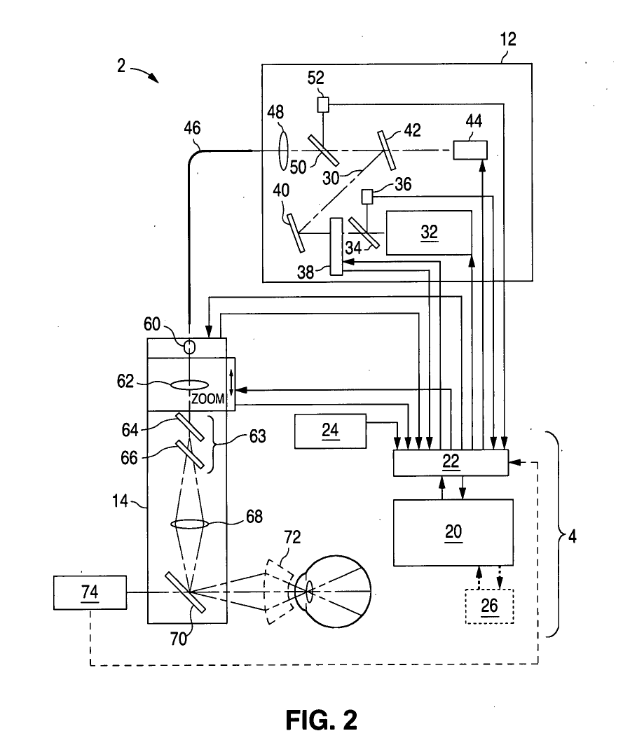

FIG. 2 illustrates an exemplary and non-limiting medical device 2 which is ideal for implementation in the above described configuration. This description is provided as an example only, for the implementation with a secondary computing device configuration described above. The medical device illustrated in FIG. 2 and described below is an ophthalmic medical device (i.e. a photocoagulator).

The photocoagulator medical device 2 illustrated in FIG. 2 includes a computing unit 4, a light generation unit 12 and a light delivery unit 14. This system can provide either pulses of therapeutic light, or continuous scans of therapeutic light, to the eye of a patient. The computing unit 4 controls the disposition (generation and delivery) of the light, and includes control electronics 20 and resident a input and output 22, as shown. Likewise, input from an input device 24 (e.g. a joystick) and/or a graphic user interface 26, may be used by the control electronics 20 for controlling the light disposition.

In the light generation unit 12, a light beam 30 is generated by a light source 32, such as a 532 nm wavelength frequency-doubled, diode-pumped solid state laser. The beam 30 first encounters a mirror 34 which serves to sample the light for safety purposes, reflecting a fixed portion towards a photodiode 36 that measures its power. Following that, the light beam 30 encounters a shutter 38, mirror 40, and mirror 42. Shutter 38 fundamentally serves to control the delivery of the light beam 30. It may be used to gate the light, in addition to grossly blocking it. Mirror 40 is configured as a turning mirror as well as a combining mirror to combine aiming light from a second light source 44 with light beam 30. The aiming light is preferable coincident along the same path as the light beam 30 to provide a visual indication of where the treatment light from source 32 will be projected onto the target tissue. After mirror 42, the light beam 30 (now including any aiming light from source 44) is directed into an optical fiber 46 via a lens 48. An optional mirror 50 can be used to direct a portion of the light beam to a second photodiode 52, which serves purposes similar to those of mirror 34 and photodiode 36 as well as a redundant monitor of the state of shutter 38. Optical fiber 46 is a convenient way to deliver the light from the light generation unit 12 to the light delivery unit 14. However, free-space delivery of the light may be used instead, especially where the light generation and delivery units 12, 14 are integrally packaged together.

In the light delivery unit 14, lens 60 conditions the light exiting the optical fiber 46. Lens 60 may be a single lens, or a compound lens. If it is a compound lens, lens 60 may be made to be a zoom lens that adjusts the spot diameter of the beam. This is useful for easily adjusting the size of patterns and their elements on the target tissue as discussed further below. An additional lens 62 may be used to image the optical beam downstream, and possible act as the zoom lens as shown. The image point of lens 62 can be done to minimize the size of optical elements downstream. A scanner 63, preferably having a pair of scanning optics (i.e. movable mirrors, wedges, and/or lenses), is used to deflect the beam 30 to form a pattern P of spots or lines (straight or curved). Preferably, the scanning optics rotate or move in orthogonal X, Y directions such that any desired pattern P can be produced. A lens 68 focuses the beam onto a mirror 70 which redirects the beam through an ophthalmic lens 72 and onto the target tissue. Mirror 70 also provides for visualization of the target tissue therethrough, either directly by the physician or by a visualization device 74. More specifically, visualization may be accomplished by directly viewing the retina through mirror 70, or by creating a video image using a visualization device 74 (e.g. CCD camera) to be displayed either on a remote monitor, or, as indicated by the dashed line of FIG. 2, on the graphical user interface 26.

Ideally, the lens 62 images the beam to a midpoint between scanning optics 64, 66 and onto mirror 70. This may be done to minimize the size of the mirror 70 in an attempt to increase the overall solid angle subtended by the visualization device 74. When mirror 70 is small, it may be placed directly in the visualization path without much disturbance. It may also be placed in the center of a binocular imaging apparatus, such as a slit lamp biomicroscope, without disturbing the visualization. Lens 62 could also be placed one focal length away from the optical midpoint of the scanning optics 64, 66 to produce a telecentric scan. In this case, mirror 70 would need to be large enough to contain the entire scan, and could be made a high reflector spectrally matched to the output of light sources 32, 44, and visualization accomplished by looking through mirror 70. Of course, a further refinement would be to photopically balance the transmission of mirror 70 by using a more complicated optical coatings to make the colors appear more natural, rather than, say, pinkish, when using a green notch filter coating on mirror 70.

Ophthalmic lens 72 may be placed directly before the eye to aid in visualization, such as might be done with any opthalmoscope, slitlamp biomicroscope, fundus camera, scanning laser opthalmoscope (SLO), or optical coherence tomography (OCT) system. Ophthalmic lens 72 may be a contact or non-contact lens, although a contact lens is preferred because it serves the additional purpose of dampening any of the patient's eye movement.

The pattern P of light formed by the scanning optics 64, 66 can be anything from a specifically located spot, an array of spots, or a continuous scan of lines or line segments. Light sources 32, 44 and/or shutter 38 may be gated on and off by commands from control electronics 20 via input and output 22 to produce discrete spots, or simply run cw to create continuous scans as a means to produce pattern P. Control electronics 20 likewise can also be configured to control the position of mirror 70 and therefore, ultimately, the pattern P. In this way, pattern P, or any of its elements may be made to be perceived by the patient as blinking. Furthermore, the perception of both discrete spots and blinking may be accomplished by simply scanning quickly between elements of pattern P to limit the amount of light registered by the patient in those intermediate spaces.

The inherent flexibility of scanned light sources thus enables many desired clinical possibilities. A device such as this may be mounted directly onto, among other things, an ophthalmic visualization tool such as a slit lamp biomicroscope, indirect opthalmoscope, fundus camera, scanning laser opthalmoscope, or optical coherence tomography system.

There are other techniques for creating pattern P, such as by moving the light source(s) directly. Alternately, scanner 63 can comprise a two-dimensional acousto-optic deflector, or one or more optical elements with optical power that are translated. Mirror 70 may be tilted or translated (if there is surface curvature) to either act as the system scanner or augment beam movement already created by scanner 63. In the case where mirror 70 has optical power, compensating optical elements (not shown) may be required to produce an image, as opposed to a simple illumination. Similarly, the beam 30 could be divided using passive elements, such as diffractive optical elements (e.g. gratings or holograms), refractive elements (e.g. beam splitters, lenslet arrays, etc), or even active devices (e.g. adaptive optics) to create multiple beams simultaneously. These beams could then be deployed at once for an even more efficient treatment. They may also be used in conjunction with scanner 63 to provide a mixed approach.

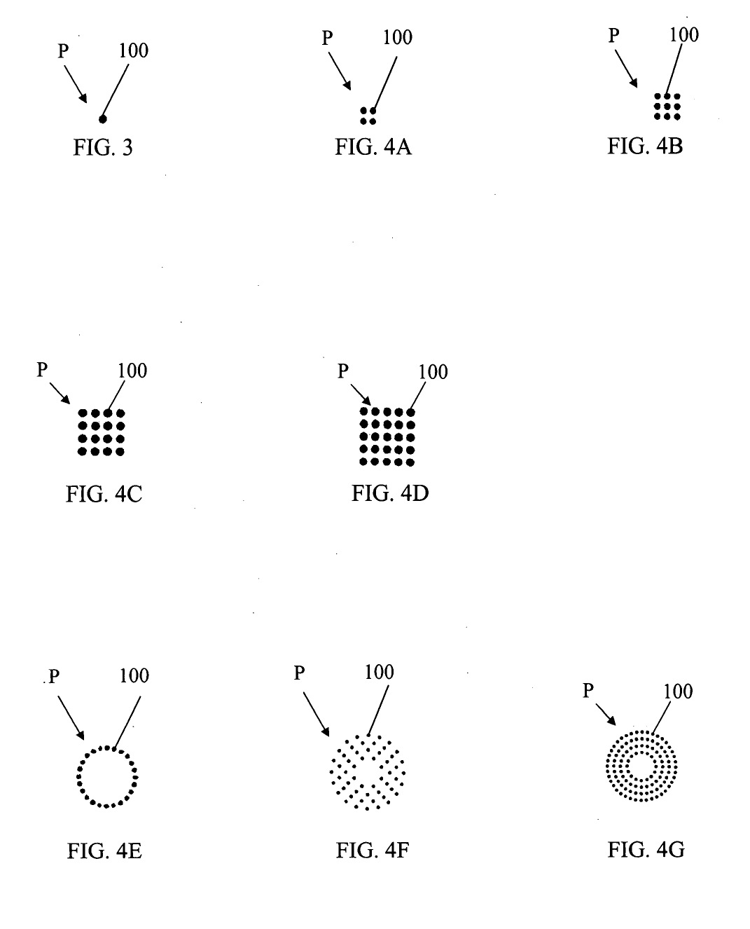

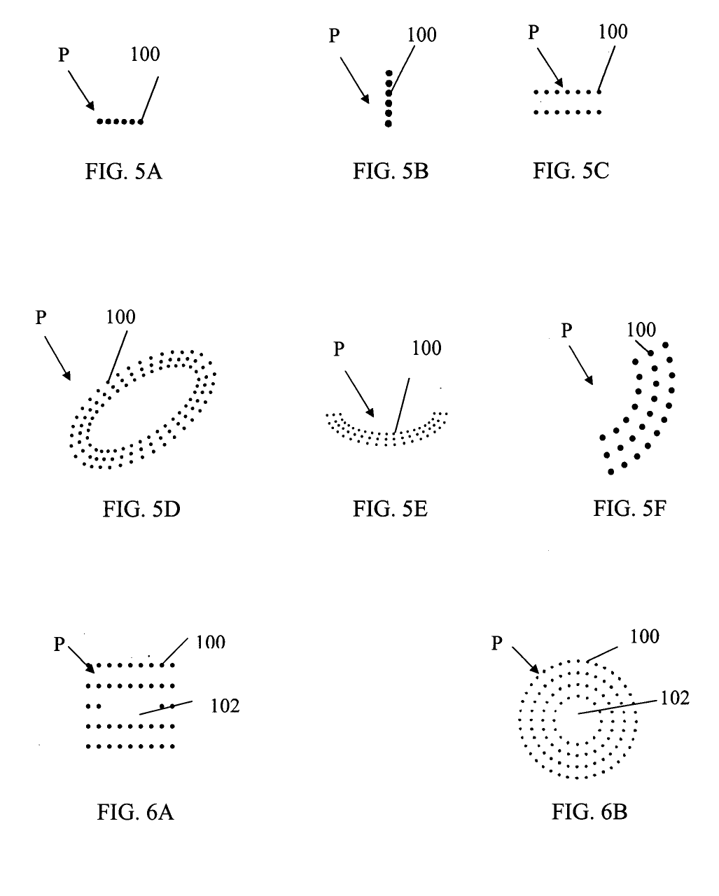

The above described system 1 is configured to provide, under the control of the computing unit 4, patterns P of pulsed or scanned light such that targeted tissue receives treatment light within specific duration ranges and locations in order to achieve the desired results. The most basic types of patterns P are those formed of discrete, uniformly sized and uniformly spaced fixed spots. The user can use GUI 26 to select, modify, and/or define a number of pattern variables, such as: spot size, spot spacing (i.e. spot density), total number of spots, pattern size and shape, power level, pulse duration, etc. In response, the control electronics 20 via input and output 22 control the treatment light source 32 (assuming it is a pulsed light source) or additionally shutter 38 to create pulsed treatment light. Mirrors 64, 66 move between pulses to direct each pulse to a discrete location to form a stationary spot. FIG. 3 shows a pattern P having a single spot 100. FIGS. 4A-4G show fully symmetrical (i.e. symmetrical in both the vertical and horizontal axes) square or circular patterns P of spots 100. FIGS. 5A-5D show non-symmetrical patterns P of spots 100 such as lines, rectangles and ellipses. FIGS. 6A-6B show patterns P of spots 100 with completely enclosed exclusion zones 102, which are zones within the pattern P that are free of spots 100. FIGS. 7A-7B show patterns P of spots 100 with partially open exclusion zones 102, where the exclusion zone 102 is not completely surrounded by the spots 100. Different patterns are ideal for different treatments. For example, a single spot pattern is ideal for titrating the power for treatment, performing touchups to space between pattern spots, and sealing individual micro-aneurysms. Rectangle, square and line patterns are ideal for PRP (panretinal photocoagulation). Elliptical and circular patterns are ideal for treating the macula, and sealing tears. Arc patterns (i.e. circular or elliptical wedge patterns but without a radially center portion as shown in FIG. 5F) are ideal for partially surrounding and treating a tear, as well as for PRP treatment for periphery and lattice degeneration. Patterns with enclosed exclusion zones are ideal for treating around sensitive areas such as the fovea where it is important that the sensitive area not receive any treatment light. Patterns with partially open exclusion zones are ideal for treating sensitive areas that are connected to other sensitive areas, such as avoiding treatment of both the fovea and the optic nerve that extends from the fovea—see especially pattern P in FIG. 7A)

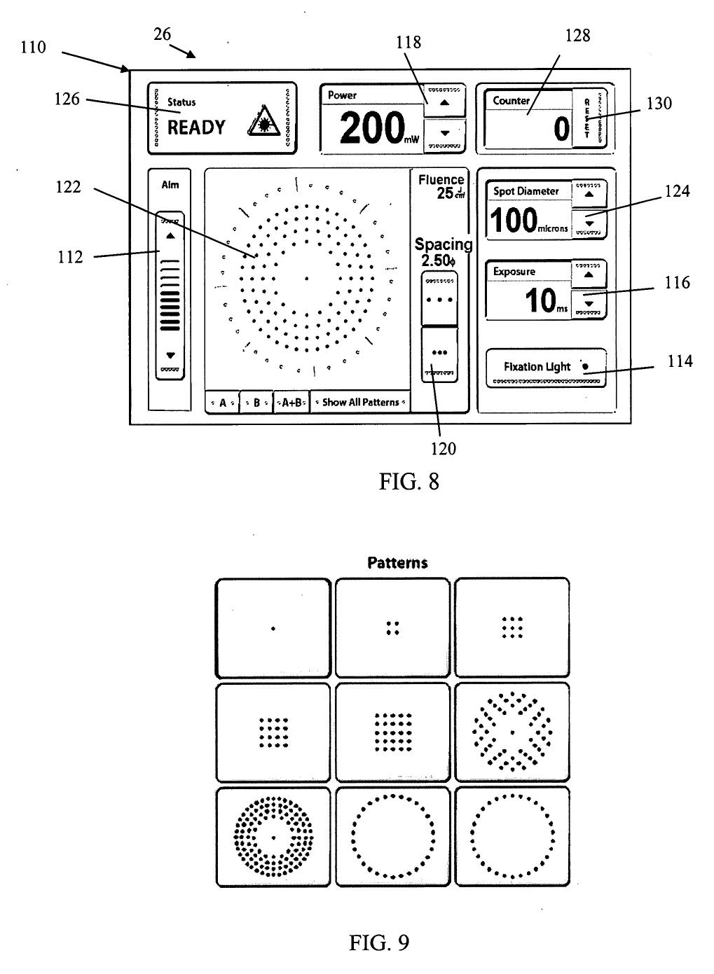

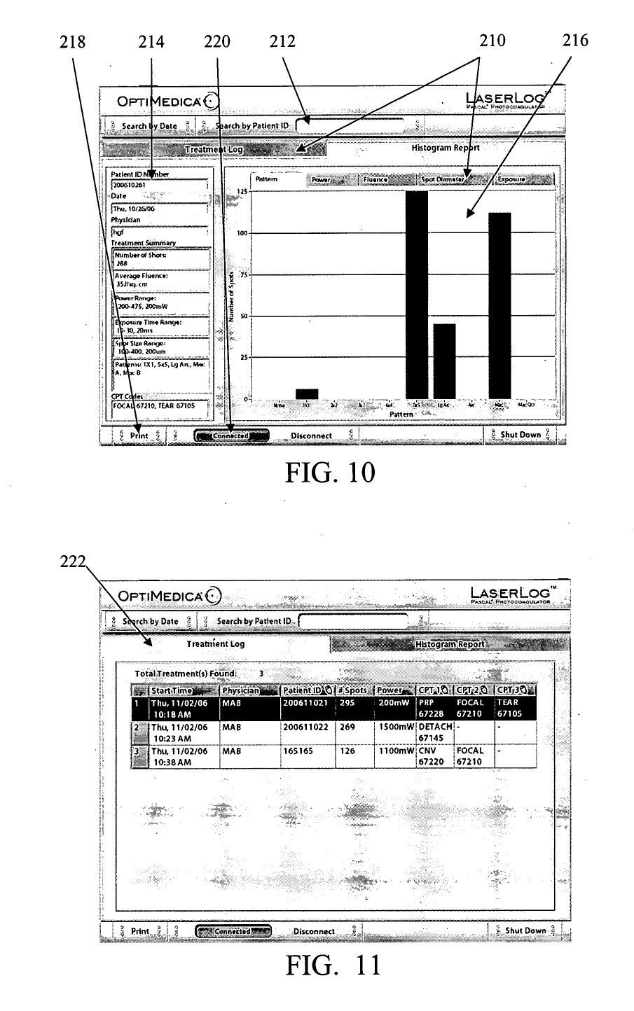

FIG. 8 illustrates an exemplary graphic user interface (GUI) 26 for selecting and implementing the above described photocoagulation patterns. The illustrated GUI 26 comprises a touch screen display 110, which defines soft keys on the screen can be used to change the operating conditions of the system. For example, the display 110 defines soft keys for adjusting aim beam power 112, fixation light power 114, exposure time 116, treatment power 118, spot density 120, pattern 122, and spot diameter 124. Touching these soft keys allows the user to adjust the selected parameter(s). Some soft keys are in the form of up/down arrows, which allow the user to directly adjust the numeric value. Other soft keys provide multiple options (e.g. spot density 120) from which the user can select the desired option. Still other soft keys illustrate an operating parameter, and when activated open new menus from which to manipulate that operating parameter (e.g. the pattern soft key 122 illustrates the configuration of the selected pattern such as spot spacing and pattern shape and layout, and when activated such as being touched opens a menu for selecting from a plurality of predefined patterns as illustrated in FIG. 9, or for defining a new pattern; the spot diameter soft key 124 indicates the size of the spots and when touched opens a menu for adjusting the spot size). Status indicators are also provided on display 110 (e.g. status indicator 126 indicates whether the system is in a standby mode, an aiming light mode, or a treatment light mode; counter indicator 128 keeps track of the number of treatment applications and can be reset by touching the reset soft key 130). Soft keys can also be tailored to the specific data being input. For example, by dragging the user's finger around pattern soft key 122 allows the user to select how many quadrants, octants, etc. that will be included in a circular pattern (e.g. dragging around the pattern key 122 for approximately 310 degrees will select a pattern with seven octants—i.e. one octant will be left out of an otherwise complete circular pattern).

Advantages of Secondary Computing Device Used With Ophthalmic Photocoagulator

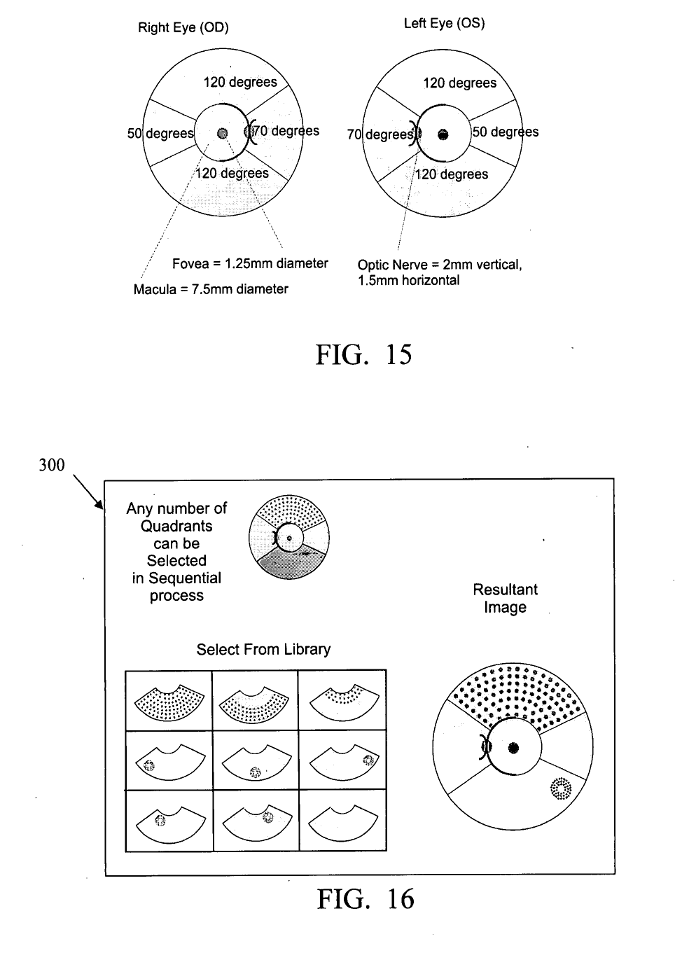

For the ophthalmic photocoagulator medical device described above, there are many advantages of providing the secondary computing device 6 in combination with a medical device having a computing unit 4:
1. The secondary computing device 6 (e.g. desktop or notebook computer, personal digital assistant PDA, etc.) preferably has its own monitor, allowing the user to view data such as a patient file without interfering with any interface or operation of the medical device 2.
2. The secondary computing device 6 can include, or have access to, an e-record database of patient specific images of the eye, which can be accessed and displayed at the treatment location for use in conjunction with slit lamp viewing to enhance the physician's ability to treat appropriately, sometimes for real-time diagnostic purposes (e.g., providing a fundus image while performing a laser photocoagulation treatment).
3. Reference materials, e-mails, videos, etc., stored or accessed by the secondary computing device 6, can be displayed on the secondary computing device 6 to aid the treatment, again without interfering with any interface or operation of the medical device 2.
4. A database/software program can be implemented by the secondary computing device 6, which could have two different modes—one for treatment information and one for servicing, where access provided to those modes can be limited by separate password protected login accounts.
5. Either at the conclusion of treatment or real-time during the treatment, treatment information can be uploaded from the medical device 2 to the secondary computing device to provide a treatment summary (including specifics such as date, time, number of laser pulses, laser parameters used, physician name, etc). This data can be combined and/or input with patient and treatment specific information such as billing information in the form of CPT codes to provide a complete report that is stored on an electronic record database and/or printed out for hard copy reports. Some of the data can auto-populate fields in the report summary and/or report detail. Since many medical devices intentionally do not have and do not want to have keyboard input device access which would open up access to the complete operating system and/or device, the input/output device 6a of the secondary computing device 6 can act as the patient/treatment input device without this drawback.
6. An automatic laser log database/file can be created/updated and accessed by the secondary computing unit 6 (e.g. via input/output device 6a). This log could also include instrument error files and preventative maintenance system check files etc. as well as treatment files. Such a database and its individual log files can be searchable by date, patient ID, system serial number, system parameter, error code, etc. The log database/file can be stored on the secondary computing unit 6, stored on a networked storage location accessible by the secondary computing unit 6, or specifically on the medical device 2 with communication enabled through the secondary computing unit 6. The log database/file could have extra security and HIPAA compliance measures in place such as encryption and password protection.
7. Laser-based medical devices are required by regulatory and industry bodies to report device usage, for example with compliance with ANSI standards set by the Laser Institute of America. The secondary computing device 6 can facilitate compliance with record maintenance on device usage with device 2 or computing unit 4 data, even assisting in the submission of records over external resource or network 8.
8. For patient records, a physician usually must document the medical procedure performed. Via the input/output device 6a, the physician can tie data from the medical device 2 or computing unit 4, and/or from external sources via external resource/network 8, as detailed below with respect to FIGS. 15-16.

FIGS. 10-11 illustrate exemplary data screens that can be accessed by the secondary computing device (e.g. via input/output device 6a) for accessing an electronic records database usable with the ophthalmic photocoagulator system described above. Dedicated soft buttons/tabs 210 and search fields 212 for navigation and display of information as outlined below provide access to information and/or data entry stored locally and/or via the medical device 2. These buttons and fields can use different search algorithms such as opening a calendar with available dates with data indicated, or free text fields, or text fields limited to a certain number or kind of character or format.

Tabs 210 organize the data on separate screens that are easily accessed. Search bar fields 212 allow the user to search database records by date or patient ID. The patient ID search can be customized to fit physician practice standards. Programmed limitations can be placed on the entry of this field such as number of characters or format of entry to protect patient privacy. Text summaries 214 are also preferably provided, which not only provide information, but also provide a convenient data entry mechanism. For example, pre-configured applicable CPT codes for treatment billing can be presented. This list can be hard coded or configurable at setup or realtime. If configurable, one method of selecting CPT codes can include activating the field (i.e. right-clicking on the field using a mouse as an input device) and selecting a code from a populated list with code descriptors and numbers.

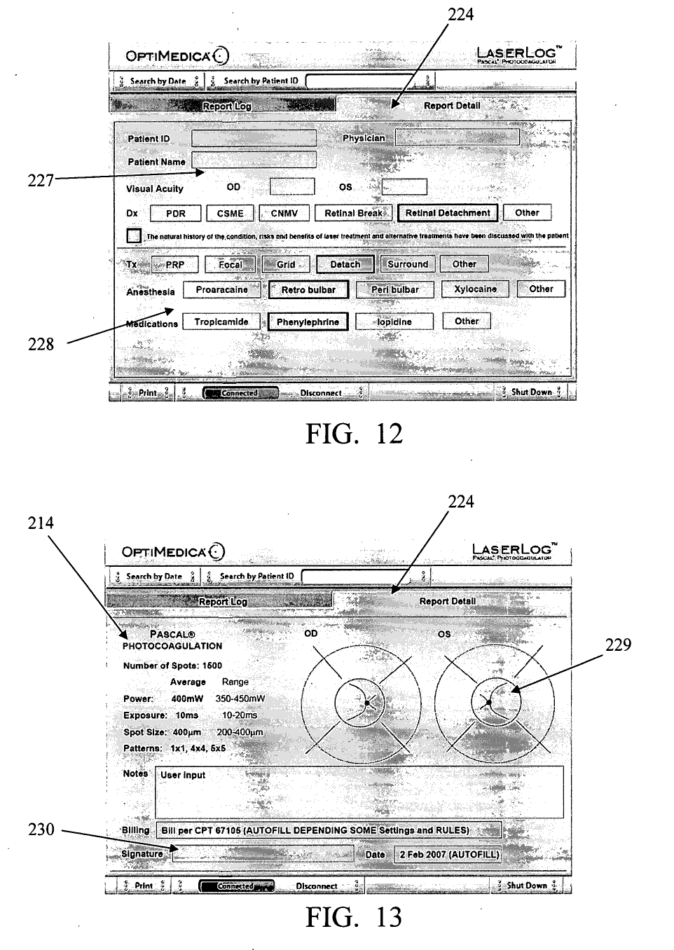

Input/output device 6a allows the physician to tie data from the medical device 2 to physiological, pathological and/or anatomical references found on the external resource/network 8. The linked data is now much more useful for the physician and corresponding records. For example, as shown in FIGS. 12-13, the treatment data 214 can be linked to diagnosis fields 227 and treatment medication fields 228. In addition, based on the treatment parameter used for pattern P, an image for pictorial reporting can be generated so the user can select an appropriate image to record the treatment (see below with respect to FIGS. 15-16). The user can correlate this graphical representation of device parameters with the illustrated anatomical position 229 without compromising the device operation. The secondary computing device 6 can facilitate compliance with record maintenance on device usage with medical device 2 and personnel authentication fields 230.

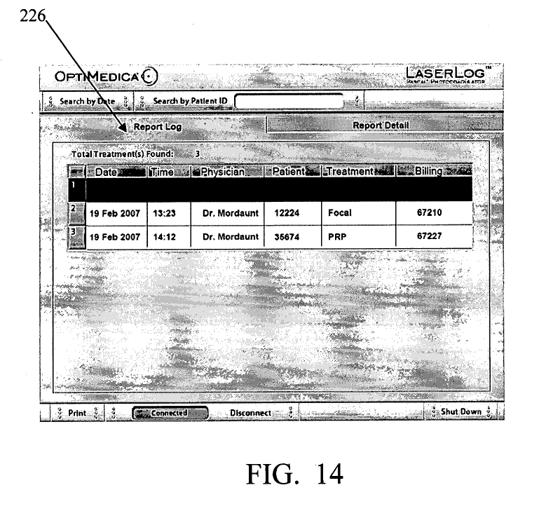

Graphic representations 216 are also preferably provided along with the text summaries 214 to allow the user to review treatment information quickly and conveniently. For example, histograms of parameters graphically present summary data to the user, where the user can dive more deeply into the data using the text summaries and other tabs (e.g. the treatment log summary tab 222 in FIG. 11) if desired for more detailed analysis or procedural reporting. A print button 218 is preferably provided, where the physician or assistant can print the treatment summary or data log of treatments using simple functionality. Preferably the printed documentation will match the values and formats on the screen for accuracy, which is easily checked by the user. A connection status tab 220 confirms that the secondary computing device 6 is connected to the medical device 2. FIGS. 12-14 illustrate further exemplary screens (report detail tab screens 224 and report log tab screen 226) provide information about the clinical procedure performed by the medical device.

The screens described above provide many benefits to physicians, such as time and effort savings by providing easy automated treatment documentation, providing compatible paper and electronic record keeping practices, providing the capacity to export or e-mail database information, providing a database that can be customized to work with existing electronic medical record systems, etc. All of these advantages result without unnecessarily tying up the computing unit 4 of the medical device 2, by providing much of the data access, data correlation from multiple sources, storage, transfer and/or printing functionality via the secondary computing device 6.

FIGS. 15-16 illustrate tools on the secondary computing device 6 that can aid the physician in generating reports documenting the clinical procedure performed using the medical device 2. FIG. 15 illustrates the various the quadrants on the eye in which patterns of spots can be used to treat the eye according to the exemplary photocoagulator described herein. FIG. 16 illustrates a screen 300 that can be provided on the input/output unit 6a, which the physician can annotate and print to summarize a recent procedure conducted using the medical device 2. The screen 300 in FIG. 16 can be passively generated by simply including a format specific to medical device 2 (i.e. include the fundamental quadrants of the eye in which treatment can take place), and then easily annotated by the physician. Alternately, screen 300 can be actively generated using data sent by the medical device 2 to the secondary computing device 6 via communications link 7, and/or data received by the secondary computing device 6 from external resource/network 8 via communications link 9 (i.e. the data can auto-populate key portions of the screen 300), and then annotated if necessary by the physician. As an example of active generation of screen 16, one or more parameters of the actual clinical procedure provided by the medical device 2 could be automatically added to screen 300, along with pathological, physiological and/or anatomical references provided from the external resource/network 8.

It is to be understood that the present invention is not limited to the embodiment(s) described above and illustrated herein, but encompasses any and all variations falling within the scope of the appended claims. For example, materials, processes and numerical examples described above are exemplary only, and should not be deemed to limit the claims. While GUI 26 is shown in FIG. 2 as connected to the medical device 2, it instead could be connected to the secondary computing device 6, and accesses the medical device over communications link 7.

What is claimed is:

1. A medical system, comprising:
a medical device that includes a computing unit for controlling the medical device to perform a clinical procedure;
a secondary computing device linked to the medical device via a first communications link;
an external resource or network linked to the secondary computing device via a second communications link;
wherein the secondary computing device is configured to provide a communications interface between the medical device and the external resource or network.

2. The medical system of claim 1, wherein the secondary computing device is a stand-alone desktop, notebook personal computer, or personal digital assistant (PDA).

3. The medical system of claim 1, wherein the secondary computing device includes an input/output unit.

4. The medical system of claim 2, wherein the input/output unit includes as least one of a keyboard, a mouse and a visual display.

5. The medical system of claim 1, wherein the external resource or network includes at least one of a data network, a printer, and a visual display.

6. The medical system of claim 1, wherein the medical device is configured to generate data and transmit the data to the secondary computing device via the first communications link, and wherein the secondary computing device is configured to transmit the data to the external resource or network via the second communications link.

7. The medical system of claim 6, wherein the secondary computing device is configured to store the data transmitted via the first communications link.

8. The medical system of claim 1, wherein at least one of the medical device and the secondary computing device is configured to generate or receive data for transmission via the first communications link.

9. The medical system of claim 8, wherein the secondary computing device is configured to transmit the data to the external resource or network via the second communications link.

10. The medical system of claim 8, wherein the secondary computing device is configured to store the data.

11. The medical system of claim 8, wherein the medical device is an ophthalmic photocoagulator that produces treatment light, and wherein the data includes photocoagulation specific data.

12. The medical system of claim 8, the data includes information about an operation by or a maintenance of the medical device.

13. The medical system of claim 8, wherein the data includes summary information about the clinical procedure.

14. The medical system of claim 13, wherein the medical device is an ophthalmic photocoagulator that produces treatment light, and wherein the summary information includes information about an application of the treatment light.

15. The medical system of claim 8, wherein the medical device is an ophthalmic photocoagulator that produces a pattern P of treatment light, and wherein the secondary computing device further comprises an input/output unit.

16. The medical system of claim 15, wherein the input/output unit comprises a search function for searching the data.

17. The medical system of claim 15, wherein the input/output unit comprises a plurality of tabs for selectively presenting portions of the data.

18. The medical system of claim 15, wherein the input/output unit comprises text summaries for presenting portions of the data in a text format.

19. The medical system of claim 15, wherein the input/output unit comprises a soft print key for printing at least a portion of the data.

20. The medical system of claim 15, wherein the input/output unit comprises a soft connection key for controlling and/or indicating a connection between the secondary computing device and the medical device via the first communications link.

21. The medical system of claim 15, wherein the input/output unit is configured to display a treatment summary for annotation via the input/output unit.

22. The medical system of claim 21, wherein the secondary computing device is configured to generate the treatment summary using data communicated from the medical device via the first communications link and data communicated from the external resource or network via the second communications link.

23. The medical system of claim 22, wherein the data communicated from the external resource or network includes at least one of a pathological reference, a physiological reference and an anatomical reference.
